(19) United States
(12) Patent Application Publication
(10) Pub. No.: US 2008/0127975 A1
(43) Pub. Date: Jun. 5, 2008

(54) Method for Preparing Gas Mixtures for Lung Ventilators and Device for Carrying Out this Method

(76) Inventors: Dominik Lirsch, Bruckneudorf (AT); Robert Kolbl, Tulln (AT)

Correspondence Address: NIXON & VANDERHYE, PC, 901 NORTH GLEBE ROAD, 11TH FLOOR, ARLINGTON, VA, 22203, US

(21) Appl. No.: 11/791,973

(22) PCT Filed: Nov. 30, 2005
(86) PCT No.: PCT/AT2005/000479

§ 371 Date: Dec. 7, 2007

(30) Foreign Application Priority Data
Dec. 1, 2004 (AT) A 2019/2004

(51) Int. Cl. A61M 16/12

(52) U.S. Cl. 128/203.25

(57) ABSTRACT

In a method for preparing gas mixtures for lung ventilators, a plurality of gases which are different from one another are supplied to a mixing chamber via switching valves, wherein the switching valves are opened and closed in a clocked manner and the pressure in the mixing chamber is monitored. The gases which are different from one another are each guided via a plurality of switching valves arranged in parallel, the switching valves being controlled in such a manner that a number of switching valves assigned to gases each different from one another, corresponding to a coarse mixing ratio is opened and closed synchronously.

The invention relates to a method for preparing gas mixtures for lung ventilators in which a plurality of gases which are different from one another are supplied to a mixing chamber via switching valves, where the switching valves are opened and closed in a clocked manner and the pressure in the mixing chamber is monitored, and the invention also relates to a corresponding device for carrying out this method.

In such methods electronically triggered switching valves are used to produce a gas mixture, which valves regulate the flow rate of the input media separately from one another and subsequently fill a mixing chamber. The flow rates of the input media are regulated depending on the timing of the switching valves determined by the switching algorithm. A defined mixing ratio of the gases at a likewise defined pressure is thus obtained in the mixing chamber.

A method of the type specified initially and a corresponding device can be deduced, for example, from U.S. Pat. No. 4,576,159. This known method uses switching valves each operated in pulsed mode. Since a high degree of pressure fluctuations occur as a result of the pulsed operation of the switching valves, it was proposed here that the gas streams guided via the valves should be supplied to an air chamber in which the pressure was equalised and correspondingly monitored. For this purpose, an elastic membrane could be provided in the air chamber or however, a valve could be provided for keeping the pressure constant, wherein the air chamber served as a mixing chamber and was primarily intended to eliminate fluctuations of the gas pressures.

Other known devices in which the switching frequency of switching valves was influenced can be deduced, for example from U.S. Pat. No. 3,626,963. U.S. Pat. No. 3,895,642 discloses the use of difference pressure valves for achieving the desired mixing ratio and finally U.S. Pat. No. 4,380,233 provides a lung ventilator with a special switching arrangement for the operation of solenoid valves whereby, in addition to the switching frequency for the pulsed valves, the ratio of the inhalation and the exhalation time could also be pre-set.

All the known devices are used to provide a mixture of two gas components and in particular, of oxygen and compressed air, with a selectable mixing ratio and, if possible, a selectable pressure. In modern jet ventilators for jet ventilation, a substantially high working pressure or emission pressure must be used to ensure sufficient gas exchange in the lung of the patient. Emission pressures of the order of magnitude of up to 4 bar are usually required here, in which case the known systems operating with proportional valves or switching valves usually regulate the gas concentration in a first work step and only then subject the desired working pressure to regulation in another work step. Such systems usually yield only inadequate pressure constancy for jet ventilation and usually only a gas flow which is too low. The flow characteristic is mostly strongly dependent on the set mixing ratio which means that the initial pressure and therefore a number of other adjustable parameters of the lung ventilator vary with the oxygen concentration.

The object of the invention is now to ensure constant mixing and pressure ratios in each case regardless of the preset mixing ratio and the amount withdrawn, which varies in each case, so that a correspondingly high flow can be supplied at a correspondingly high pressure to a jet ventilator.

In order to achieve this object, the method according to the invention substantially consists in that the gases which are different from one another are each guided via a plurality of switching valves arranged in parallel and the switching valves are controlled in such a manner that a number of switching valves assigned to gases each different from one another, corresponding to a coarse mixing ratio are opened and closed synchronously. Since a plurality of valves are arranged in parallel in each case for different gases such as oxygen and air, for example, by selecting the corresponding number of valves to be opened in each case, it is possible to roughly ensure a desired mixing ratio and furthermore a desired flow rate. These valves or valve groups used for the basic setting in each case can accordingly be operated synchronously in a clocked manner, where the respective gas streams are supplied to the mixing chamber. The mixing chamber thus directly receives the desired mass of gas per unit time and the respective gas streams in the desired mixing ratio. In order to now ensure fine tuning to the desired mixing ratio, without hereby impairing a respectively predetermined flow rate, the method according to the invention is advantageously further developed such that at least one further switching valve used for matching to a defined mixing ratio is opened and closed asynchronously compared to other additional switching valves, wherein the sum of the open other valves in each case is kept constant. This further switching valve or these further switching valves are now operated asynchronously so that if, for example, a valve in the oxygen line is closed, a valve in the air line or another gas line is opened. As a result of this alternating operation, the overall gas flow rate can be kept constant and nevertheless the desired mixing ratio can accordingly be exactly set. For this purpose, it is advantageous to proceed such that the total number of open valves in each case is selected depending on the desired flow rate and is kept constant. The switching valves operated in clocked or pulsed mode in each case can be operated in a pulsed mode according to the requirements of the pressure regulations, in which case the pressure itself can be subjected to a two-point regulation in a conventional manner and the corresponding control signals can be provided directly to the switching valves. Gas can be branched off via a throttle valve and fed to a gas analyser, whereby the mixing ratio can be checked continuously and a quality control is possible.

The device according to the invention for carrying out this method is substantially characterised in that between each connection for a gas line and the mixing chamber, there is incorporated in each case a plurality of switching valves capable of being opened and closed in a clocked manner, and that the switching valves are connected to a controller and a clock generator, whose clock frequency or control signals for opening and closing the switching valves can be adjusted separately depending on specifications for the mixing ratio of the gases and the desired flow rate for individual valves in the same gas path. In order to ensure the corresponding constant pressure and the corresponding mixing ratios at a correspondingly high flow rate, the device is advantageously configured such that at least three, preferably four, valves are arranged parallel to one another in each gas path, and the mixing chamber is preferably configured as an air chamber whose pressure is monitored by at least one sensor from which sensor signals are supplied to a controller for pressure regulation.

For quality control purposes, the device is advantageously configured such that in at least one gas path there is provided a throttle valve to a branch line to which a gas analyser can be connected.

Overall, the configuration according to the invention provides a device in which the corresponding pressure, flow rates and mixing constancy can be preset in one working process so that a substantially smaller mixing chamber can be used to equalise the pressure peaks. The mixing ratio can be adjusted with higher accuracy with a deviation of less than 1% from the desired value, whereby corresponding buffering in the mixing chamber can be ensured in cases of short-term maximum withdrawal.

The invention is now explained in detail with reference to an exemplary embodiment shown schematically in the drawings. In these,

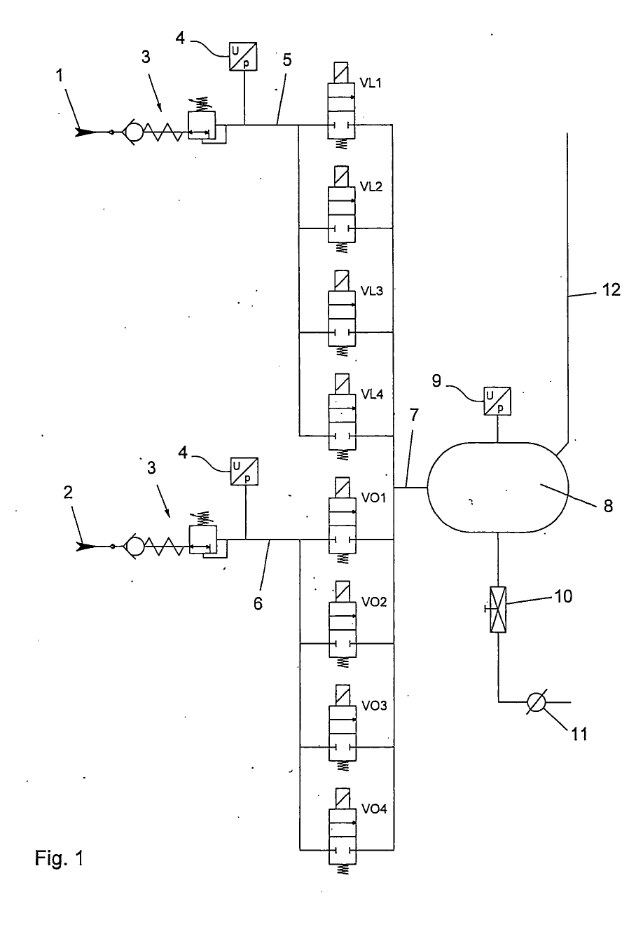

FIG. 1 is a schematic diagram of the device according to the invention.

FIG. 2 is a diagram for switching times of the valves for setting the desired mixing ratio and

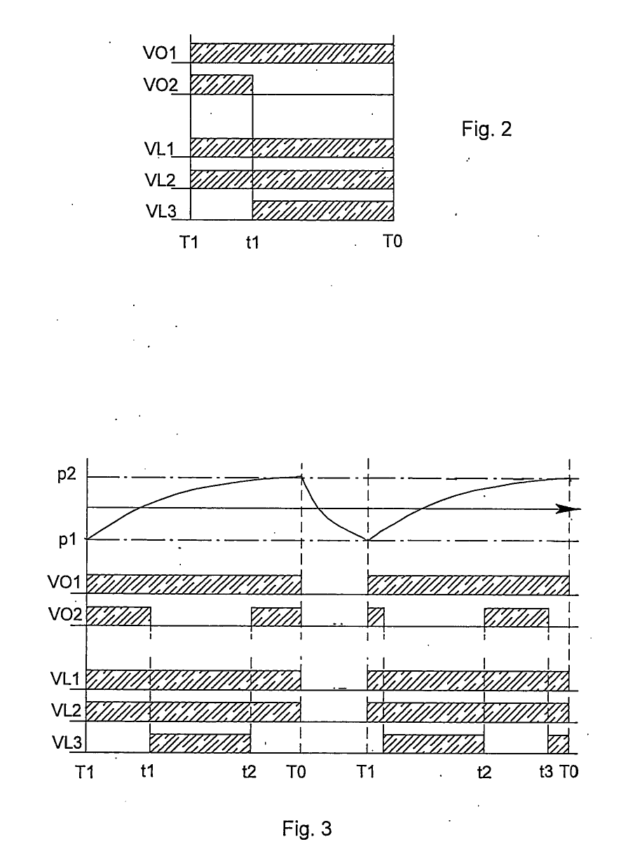

FIG. 3 is a modified and supplemented diagram according to FIG. 2 in which the pressure profile and thus the pressure regulation is shown over a plurality of switching cycles.

In FIG. 1, the compressed air coupling is indicated by 1 and the oxygen coupling of the corresponding gas supply of a hospital is indicated schematically by 2. Pressure regulators 3 are connected after each of the respective couplings, the corresponding pressure sensors being designated by 4. In each case, a plurality of valves are arranged in parallel in the respective gas paths 5 and 6, four valves being provided in each case in the diagram according to FIG. 1, which are designated as VL1 to VL4 in the case of the air path and are designated as VO1 to VO4 in the case of the oxygen path. All these valves are configured as valves which can be opened and closed in a clocked manner and are opened and closed in each case by a pulse generator not shown in detail. The respective total output of the gas lines 5 and 6 is combined via a line 7 and supplied to a mixing chamber 8. The mixing chamber 8 can be relatively small, in which case the gases supplied at high pressure and in suitable quantities can be separated from one another for better turbulence and can be fed tangentially into the mixing chamber. The mixing chamber itself has a pressure sensor 9 by which means the respective upper or lower pressure value for the two-point regulation is set. Reference numeral 10 designates a throttle valve via which gas is supplied to an oxygen sensor 11 for analysis of the gas. The mixture is supplied to the lung ventilator via the line 12.

In the representation according to FIG. 2 a diagram can now be seen in which a possible operating mode for four valves for each of the oxygen and the air paths is shown schematically. The oxygen valve VO1 like the two air valves VL1 and VL2 is open over the entire time interval from T1 to T0. At the same switching frequency and the same flow cross-sections, an air:oxygen mixing ratio of 2:1 is obtained, where T1 indicates the switch-on time and T0 is the respective switch-off time. Additional valves V02 and VL3 are provided for fine adjustment of the mixing ratio, these being now opened and closed alternately, i.e. operated asynchronously, so that overall in each case from T1 to T0 that flow rate which would correspond to a further valve is implemented since the two valves VO2 and VL3 are switched over at time t1 so that in each case only one valve is always open. The time interval of the valve change produces a corresponding fine adjustment of the mixing ratio and in particular the desired oxygen concentration in the air-oxygen mixture.

In the diagram in FIG. 3 the corresponding pressure profile can additionally be seen over the respective switching times of the various valves VO1, VO2 as well as VL1, VL2 and VL3. At the switch-on time T1 the pressure is below the lower limit p1 and increases in the course of the switch-on cycle which also contains the respective valve changeover times t1 and t2 for switching over the valves, as far as a pressure p2 whereupon at this time T0 a switch-off is effected until the pressure has dropped from p2 to p1 again. As a consequence, pressure values between p1 and p2 are achieved, correspondingly shifted from T1 to T0, where three switching changeover times t1, t2 and t3 are now observed in order to ensure the desired constancy and also the desired mixing ratio for the same mass per unit time.

The switching valves V01 to V04 or VL1 to VL4 fill the mixing chamber with the desired mixture in each case, all the valves being closed as soon as the upper pressure limit is reached in the mixing chamber. As soon as the pressure drops to the lower limit, the corresponding valves are again opened in each case whereby two-point regulation is implemented. The number of switching valves used for air and oxygen predefines a coarse mixing ratio, and the mixing can be additionally refined by briefly switching to and fro at least one air and one oxygen valve whereby the resulting oxygen concentration in the mixing chamber can be set with a high accuracy of less than +/−1 wt. %.

The central gas supply of a hospital usually delivers air and oxygen at pressures between 4 and 10 bar, the respective operating pressures usually being set to 4.5 to 5 bar by means of an input pressure regulator. Even when it cannot be ensured, for example, that a central gas supply of a hospital reaches 4.5 to 5 bar or more, safe operating modes can still be achieved with restrictions, such as for example reduced flow or emission pressure so that the operating pressures after the input pressure regulators for air and oxygen should be exactly the same for an optimum air-oxygen mixture.

In the mixing chamber the oxygen concentration is measured by means of an oxygen sensor which is connected to the mixing chamber via a controllable choke pressure regulator. The controller records signals from all the specified sensors and in particular the signals from the sensors 4 of the input regulator 3 to take into account the different operating pressures in the gas supply. If the pressures in the air or oxygen line are not symmetrical, pressure differences of up to 1 bar can be compensated by suitably influencing the clock frequencies or opening times of the respective valves VL1 to VL4 or VO1 to VO4.

1. A method for preparing gas mixtures for lung ventilators in which a plurality of gases which are different from one another are supplied to a mixing chamber via switching valves, wherein the switching valves are opened and closed in a clocked manner and the pressure in the mixing chamber is monitored, characterised in that the gases which are different from one another are each guided via a plurality of switching valves arranged in parallel and the switching valves are controlled in such a manner that a number of switching valves assigned to gases each different from one another, corresponding to a coarse mixing ratio are opened and closed synchronously.

2. The method according to claim 1, characterised in that one further switching valve used for matching to a defined mixing ratio is opened and closed asynchronously compared to other additional switching valves, wherein the sum of the open other valves in each case is kept constant.

3. The method according to claim 1, characterised in that the total number of the actually open valves is selected depending on the desired flow rate and is kept constant.

4. A device for preparing gas mixtures for lung ventilators in particular for carrying out the method according to claim 1, comprising at least two connections for gases which are different from one another, which are connected to a mixing chamber via switching valves, wherein the switching valves can be opened and closed in a clocked manner, characterised in that between each connection for a gas and the mixing chamber, there is incorporated in each case a plurality of switching valves arranged in parallel and capable of being opened and closed in a clocked manner, and that the switching valves are connected to a controller and a clock generator, whose clock frequency or control signals for opening and closing the switching valves can be adjusted separately depending on specifications for the mixing ratio of the gases and the desired flow rate for individual valves in the same gas path.

5. The device according to claim 4, characterised in that the clock frequency or the control signals can be adjusted in such a manner that a number of switching valves assigned to gases which are each different from one another, corresponding to a coarse mixing ratio of the gases can be opened and closed synchronously.

6. The device according to claim 4, characterised in that the clock frequency or the control signals are adjustable in such a manner that at least one further switching valve used for matching to a defined mixing ratio is opened and closed asynchronously compared to other additional switching valves, wherein the sum of the open other valves in each case is kept constant.

7. The device according to claim 4, characterised in that at least three, preferably four valves are arranged parallel to one another in each gas path.

8. The device according to claim 4, characterised in that the mixing chamber is configured as an air chamber whose pressure is monitored by at least one sensor from which sensor signals are supplied to a controller for pressure regulation.

9. The device according to claim 4, characterised in that in at least one gas path there is provided a throttle valve to a branch line to which a gas analyser can be connected.
